(19) United States
(12) Patent Application Publication
(10) Pub. No.: US 2012/0178671 A1
(43) Pub. Date: Jul. 12, 2012

(54) INHIBITION OF NFK-B MEDIATED VIRUS REPLICATION WITH SPECIFIC OLIGOSACCHARIDES

(75) Inventors: Klaske Van Norren, Renkum (NL); Francina Jeannette Dijk, Renkum (NL); Benjamin Berkhout, Naarden (NL); Belinda Van't Land Potappel, Kootwijk (NL)

(73) Assignee: N.V. Nutricia

(21) Appl. No.: 13/378,922

(22) PCT Filed: Jun. 21, 2010
(86) PCT No.: PCT/NL2010/050380

§ 371 Date: Mar. 2, 2012

(30) Foreign Application Priority Data
Jun. 19, 2009 (NL) PCT/NL2009/050363

(51) Int. Cl. A61K 31/717; A61P 31/12; A61P 31/18; A61K 38/02

(52) U.S. Cl. 514/3.7; 514/57

(57) ABSTRACT

The inventors surprisingly found that specific oligosaccharides are capable of inhibiting viral replication through inhibiting NF-κB activation. The invention thus pertains to a composition comprising pectin (in the form of digalacturonic acid, trigalacturonic acid, polygalacturonic acid), Arabinoxylan from rice bran, β-glucan from bakers yeast, D-Ribose or mixtures there-of for inhibiting viral replication in a mammal with a viral disease.

### BACKGROUND

The present invention is in the field of virus replication, for treating NFK-B mediated viral infections, particularly HIV infection.

Gut-derived bacterial products such as lipopolysaccharides (LPS) and peptidoglycans (PG) contribute to hyper-immune activation resulting in increased disease progression of HIV-1 infection. This hyper-immune activation might be due to chronic LPS or PG induced Toll-like receptor (TLR)-mediated NF-κB activation, leading to increased HIV-1 replication. Dietary Acidic Oligosaccharides from pectin hydrolysate (pAOS) have been suggested to inhibit adhesion of bacterial pathogens in the gastrointestinal tract.

WO 2009/0976772 [publication date 6 Aug. 2009] discloses a nutritional composition comprising dietary fiber for stimulating neutral killer cell activity, in particular comprising a mixture of galactooligosaccharides, fructooligosaccharides and pectin degradation products.

US 2006/0178341 discloses the use of a soluble glucan oligomer derived from a yeast variant strain for the preparation of therapeutic agent for treatment and prevention of diseases caused by the infection of influenza virus and transmissible gastroenteritits coronavirus. Its action is based on increased nitric oxide (NO) production. It is silent on viral replication effects of beta-glucan.

WO 02/47612 discloses a dietary supplement composition for promotion of good health and immune system support in general. Components such as beta-glucan are suitable candidates as potent regulators of the immune system. For beta-glucan, reference is made to studies on cancer cell growth and metastasis, and bacterial infection. Pectin is for instance linked with lowering of blood cholesterol, and inhibition of tumor development. Again, the document is silent on viral replication

### SUMMARY OF THE INVENTION

The inventors surprisingly found that specific oligosaccharides are capable of inhibiting viral replication through inhibiting NF-κB activation. The attached experimental data show that in an in vitro model the Toll like receptor induced and the TNF-α induced NF-κB stimulation can be inhibited. A large group of compounds was tested in further experiments, but it shows that the production of human immunodeficiency virus was inhibited with few specific oligosaccharides.

Therefore a preferred embodiment according to the invention is the use of oligo- and/or polyuronic acids, arabinoxylan (preferably from rice bran), β-glucan, preferably β-1,3/1,6-glucans such as derived from yeasts, D-ribose, and/or mixtures thereof, for inhibiting viral replication in a mammal with a viral disease, wherein the viral disease is preferably selected from the group consisting of AIDS (HIV)—AIDS Related Complex, Chickenpox (Varicella), common cold, cytomegalovirus Infection, colorado tick fever, dengue fever, ebola hemorrhagic fever, hand, foot and mouth disease, hepatitis, herpes simplex, herpes zoster, herpes Papiloma Virus, influenza (Flu), lassa fever, measles, marburg hemorrhagic fever, infectious mononucleosis, mumps, norovirus, poliomyelitis, progressive multifocal leukencephalopathy, rabies, rubella, SARS, smallpox (Variola), viral encephalitis, viral gastroenteritis, viral meningitis, viral pneumonia, west nile disease, yellow fever.

In one preferred embodiment, the invention pertains to a composition comprising one or more oligo- and/or polyuronic acids, Arabinoxylan (preferably from rice bran), β-glucan from bakers yeast, D-Ribose, or mixtures thereof, (suitable) for inhibiting viral replication in a mammal with a viral disease as disclosed above.

Also, the invention pertains to the use of one or more of:
polyuronic acids, preferably digalacturonic acid, trigalacturonic acid and/or polygalacturonic acid (preferably pectin comprising digalacturonic acid, trigalacturonic acid and/or polygalacturonic acid),pectin comprising or in the form of digalacturonic acid, trigalacturonic acid and/or polygalacturonic acid,
Arabinoxylan, preferably from rice bran,
β-glucan from bakers yeast, preferably β-1,3/1,6-glucans such as derived from yeasts,
D-Ribose,

and/or mixtures for the manufacture of a composition for inhibiting viral replication in a mammal with a viral disease as disclosed above.

Also, the invention pertains to a method of treating a person suffering from viral disease, or at risk thereof, said method comprising administering to said person the aforementioned composition.

In a preferred embodiment, the composition comprises polyuronic acids, preferably digalacturonic acid, trigalacturonic acid and/or polygalacturonic acid, and cysteine equivalents, preferably N-acetyl cysteine (NAC), preferably having a weight ratio of polyuronic acid and cysteine equivalents between 10:1 and 1:10, more preferably 5:1-1:5. Alternatively or additionally, the composition comprises 100 mg-10 g of polyuronic acids, preferably pectin AOS per daily dosage. The same weight amounts and ratios are preferred in case of other saccharides defined in the description. All saccharides preferably have chain length having 2-250 units. These can be derived from natural sources, if necessary by acid or enzymatic hydrolysis as known in the art.

In a preferred embodiment, the composition comprises 3 polyunsaturated fatty acids, preferably at least EPA, either alone or in combination with one or more cysteine equivalent(s).

### LIST OF FIGURES

FIG. 1 The effect of chain length of galacturonic acid oligosaccharides on NFkappaB inhibition. For inhibition by a polygalacturonic acid oligosaccharide a mimimal chain length of 2 galacturonic acid moieties is needed. The best effect is obtained with oligosaccharides comprising at least 3 galacturonic acid moieties;

FIG. 2. NFkappaB response in HEK293 cells after 22.5 hr. incubation with AOS and NAC (A) or L-cysteine (B) with TNF-α for 2.5 hr. Upon correction for the contribution of NAC or cysteine, the curves C and D are obtained, showing that the components are strictly additive;

FIG. 3. Glutathione concentration at different NAC and AOS concentrations. The effect of the combination is more than the effect of the separate components over the complete curve. In the figures, “AOS” stands for pAOS.

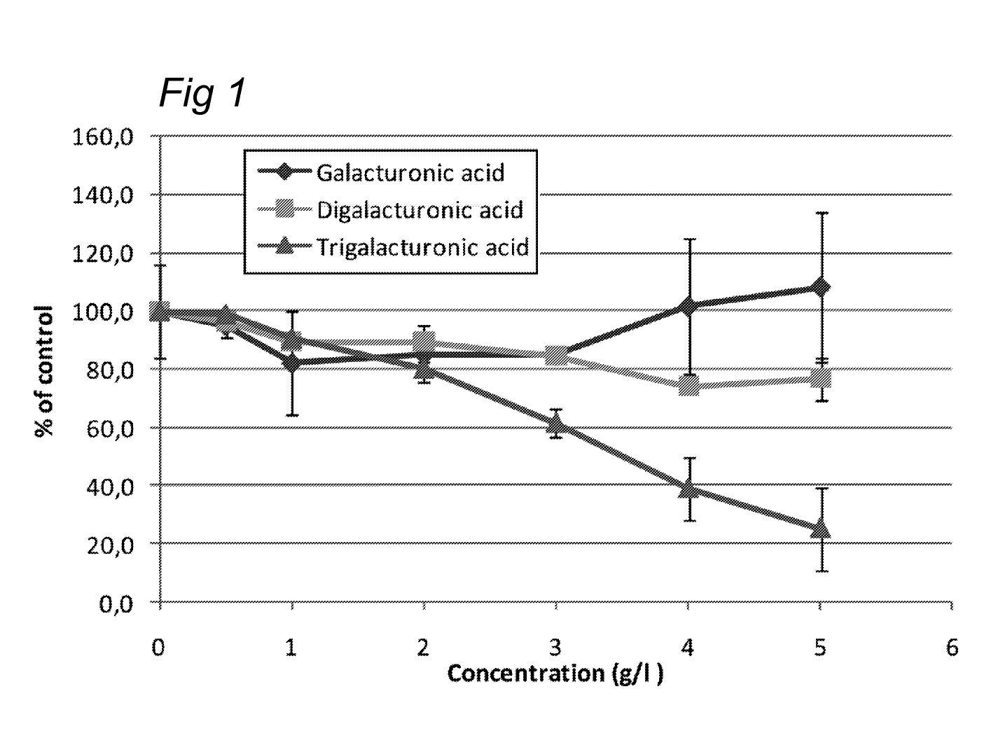

### DETAILED DESCRIPTION OF THE INVENTION

CD4⁻ T cell count is a good indicator for AIDS progression, since HIV infects primarily CD4⁺ helper T cells, eventually leading to cell death. Chronic immune hyperactivation, a typical feature in progressive HIV disease, plays a crucial role in ongoing CD4+ T cell depletion. Brenchley et al. Microbial translocation is a cause of systemic immune activation in chronic HIV infection. Nat Med. 2006;12:1365-1371. have proposed a major role for the gut in HIV-induced chronic immune activation. Increased gut permeability is common in HIV-infected patients and causes translocation of microbial products. Circulating lipopolysaccharide (LPS), a reliable marker of microbial translocation, has proved to be significantly raised in HIV-infected patients, correlating with immune hyperactivation. Moreover, LPS levels were decreased upon treatment with HAART. LPS is a major component of gram-negative bacterial cell walls and therefore a potent immunostimulatory product. These findings suggest that HIV infection causes impaired gut barrier function, followed by increased microbial translocation that eventually contributes to chronic immune activation. HIV replication seems to play a central role in maintaining this impaired gut barrier function.

The cause of gut permeability upon HIV infection is not completely understood, although it might be a result of CD4⁻ T cell infection. The GI tract is probably the largest lymphoid organ in the body, accounting for at least half of the total T cell load. Because CD4⁻ T cells are most abundant in the GI-tract, HIV infection creates a pro-inflammatory environment in the gut. It is hypothesized that this environment establishes a breakdown of the gastrointestinal mucosal barrier, and thereby augmenting microbial translocation. These bacterial products, like lipopolysaccharides and peptidoglycans (PG), induce an inflammatory response via binding to TLR receptors, which not only induces inflammation, but also triggers the HIV virus into replication. The released HIV particles infect other CD4⁺ T cells, which creates an even greater pro-inflammatory environment and increases gut permeability. This positive feedback-loop drives chronic immune activation.

Without being bound by theory, the inventors hypothesize that hyperimmune activation is mediated through LPS- or PG-induced toll-like receptor (TLR) activation. Each toll-like receptor subset is stimulated by a specific microbial product; LPS stimulates the TLR-4, while PG stimulates the TLR-2. The activation of TLRs induces a signaling pathway whereupon NF-κB will be activated, leading to inflammation. NF-κB is a dimer composed of two proteins of the Rel/κB family of transcription factors, which are both required for DNA binding. NF-κB is constitutively expressed, but is present as an inactive complex with the IκB inhibitor protein in the cytoplasm of unstimulated cells. When NF-κB is released from its complex with IKB, it is free to translocate to the nucleus where it functions as a transcription factor. Phosphorylation of NF-κB increases nuclear localization, protein-protein interactions, and transcriptional activity. When leukocytes are infected with HIV-1, NF-κB translocation to the nucleus plays an additional role in virus replication. NF-κB recognizes two 10bp stretches of DNA with the consensus sequence 5′-GGGPuNNPuPuCC-3′ in the long terminal repeat (LTR) of HIV-1. In transient transfection studies, mutation of either KB sites resulted in a dramatic loss of LTR promoter activity, suggesting an essential role for NF-κB in HIV replication. Due to its this role, NF-κB is a potential target for treatment of HIV-infected individuals. Indeed, it was found that inhibition of NF-κB activation decreases HIV-1 replication in vitro (see examples), and may therefore postpone AIDS development in vivo.

It is hypothesized that the main site of action of the specific nutritional oligosaccharides as mentioned in example 1, is the intestine. Example 1 shows a large group of dietary fibers that were tested for the NF-κB inhibiting capacity. It was found that only a small selection comprising acid oligosaccharides (shown for pectin (in the form of digalacturonic acid, trigalacturonic acid, polygalacturonic acid)), Arabinoxylan from rice bran, β-glucan from bakers yeast, D-Ribose were capable of inhibiting NF-κB in the present test system.

In the case of HIV infection, the gut is known to have high levels of viral replication in associated CD4+ T-lymphocytes during the acute and chronic phases of infection. It is further known that the gut is seen as a separate compartment that is difficult to reach with conventional anti retro viral therapy (e.g. blood viral levels can be reduced by antiretroviral therapy without influencing viral load in the intestine). The supposed ability of dietary oligosaccharides to modulate different pathways of NF-κB activation leading to reduced viral production especially in the gut designates a potential beneficial role for dietary oligosaccharides in the management of HIV infection. It may result in a reduced viral load in the gut, which might systemically translate into either a reduction of viral load or an increase in CD4 count or a combination of both.

As a result of the reasoning above it could be that the viral load in the peripheral blood of the patients is not directly altered by a treatment with NF-kB inhibitors such as AOS and/or NAC. This may be explained by the compartmentalization of the gut, where these nutritional ingredients are likely to have their strongest effect

Anti-Viral Medicaments

The supposed property of targeting a compartment difficult to address by anti retroviral therapy also implicates that the use of these specific oligosaccharides are likely to act synergistically with current anti viral therapies and in particular with an anti retroviral therapy (HAART). Due to the NF-κB inhibiting activity of the oligosaccharides according to the invention, a preferred composition according to the invention comprises a combination of anti viral therapy (e.g. HAART) with the compositions according to the invention. For the treatment of HIV the compositions according to the claims may be used in combination with anti viral vaccines, with antiviral pharmaceuticals like oseltamivir (Tamiflu), zanamivir (Relenza) and the like, or anti-retroviral therapeutics used for treatment of AIDS such as nucleoside and nucleotide reverse transcriptase inhibitors (nRTI), non-nucleoside reverse transcriptase inhibitors (NNRTI), protease inhibitors (PIs), integrase inhibitors such as Raltegravir, entry inhibitors (or fusion inhibitors) such as Maraviroc and Enfuvirtide, or maturation inhibitors such as bevirimat and Vivecon.

Composition

Preferably the present composition comprises uronic acid oligo- and/or polysaccharide, preferably oligo- and/or polygalacturonic acids. Most preferably, these are pectin hydrolysates, in the art typically addressed as pectin acid oligosaccharides (pAOS). Most preferably pectin (hydrolysate) is present in the form of or comprising digalacturonic acid, trigalacturonic acid, polygalacturonic acid. The uronic acid oligosaccharides, preferably pectin hydrolysates, are preferably obtainable or obtained by enzymatic digestion of pectin with pectin lyases, pectate lyase, endopolygalacturonase and/or pectinase.

In the context of the invention, the terms “oligouronic acid” and “polyuronic acid” are used interchangeably, and the oligo- and polyuronic acids are understood to have a DP in the range of 2 to 250, more preferably a DP of 2 to 100, even more preferably a DP of 2 to 50, most preferably a DP of 2 to 20. The uronic acid oligo- and polysaccharides are preferably galacturonic acid oligo- and polysaccharides (including digalacturonic acid, trigalacturonic acid). Most preferably, the degree of polymerisation is an average DP. In one embodiment, the composition comprises polygalacturonic acid.

Cysteine or Source of Cysteine

The inventors found that N-acetyl cysteine (NAC) is also capable of blocking NF-κB activation. The mechanism behind this suppressive capacity has not been cleared, but it has been hypothesized that it involves NAC's ability to decline oxidative stress indirectly via GSH level recovery. Oxidative stress has shown to promote NF-κB activation, via a mechanism that is still debated and probably cell-type specific. However, most studies state ROS-induced NF-κB activation to be dependent on the classical pathway by phosphorylating IKK indirectly via PKD (Protein Kinase D) activation.

Because the inventors found that NAC and AOS act together on NF-κB activation, it was hypothesized that NAC and AOS also have additive effects on GSH levels. To determine this, HEK293 cells were incubated with AOS for 22.5 hr. and NAC in combination with TNF-α for 2.5 hr. Obtained GSH levels were corrected for cell numbers and results were compared levels with TNF-α-stimulated cells (control). The addition of NAC enhances GSH levels in a concentration of 10 mM. AOS is able to significantly increase GSH levels after addition of 2 and 3 mg/l. The combination of NAC and AOS enhance GSH production up to 278% compared with control. Data sets were compared with their baseline controls (0 mg/ml AOS in combination with its specific concentration of NAC or L-cysteine) and presented in example 2. High concentrations of AOS and NAC result in a relative stronger increase in GSH levels. From this, it is concluded that AOS and NAC have synergistic effects on the production of GSH in HEK293 cells.

The compositions provided comprise in addition to one or more oligosaccharides as described above a suitable amount of cysteine and/or source of cysteine. The phrase “source of cysteine” refers herein to all compounds that contain a biologically available cysteine, in any form, and is calculated as the amount of cysteine amino acid that is present in a compound, or can be derived from a compound in the body after ingestion, on a molar basis.

Hereinbelow “cysteine equivalent” refers to an amount of cysteine as such or to an amount of cysteine that is present in a source of cysteine. For example 100 mg NAC (N-acetylcysteine; MW=163.2) is equivalent to 74 mg cysteine (MW 121.15). Thus 100 mg NAC is 74 mg cysteine equivalent. Similarly this can be applied to proteins or peptides. When a peptide (MW=×Dalton) contains 3 cysteine amino acids (3yDalton), than 100 mg of this peptide is equivalent to 100×3Y/X mg cysteine. Thus 100 mg of this peptide is 300y/x mg cysteine equivalent.

Suitable sources of cysteine according to the invention are, for example, proteins in denatured and/or undenatured form such as milk proteins e.g. whey or casein proteins. Egg proteins are rich in cysteine and are therefore also suitable. Plant proteins such as pea, potato, soy and rice can also be used to provide cysteine. Also hydrolysates of these protein sources can be used or fractions enriched for cysteine rich proteins or peptides (e.g. as described in EP1201137). Furthermore, synthetic cysteine equivalents, e.g. derivatives of cysteine, such as cysteine, cystine, cysteine salts, N-acetylcysteine and/or diacetylcysteine can be used.

The HIV infected subjects are suitably administered a daily dose of at least about 100 mg cysteine equivalent, preferably at least about 200, 400, or 600 mg cysteine equivalent per day, more preferably at least about 1000 mg cysteine equivalent per day. It is understood that a daily dosage can be subdivided into 2, 3 or more dosage units taken several times a day.

In yet another embodiment the compositions according to the invention comprise one or more compounds that stimulate glutathione levels. e.g. lipoic acid, pyruvate, oxaloacetate, oxaloaspartate, are capable in stimulating glutathione levels. Such glutathione level stimulating compounds may be used in addition to cysteine but also instead of cysteine.

ω-3 Polyunsaturated Fatty Acids

The inventors found that ω-3 polyunsaturated fatty acids are also capable of blocking NF-κB activation. This is demonstrated in the accompanying examples, where an effect is shown only in case such polyunsaturated fatty acids are combined with AOS and cysteine. The compositions provided preferably comprise in addition ω-3 polyunsaturated fatty acids. The unsaturated fatty acid for a treatment in accordance with the invention is preferably selected from the group of C18-C26 ω-3 polyunsaturated fatty acids. Preferred ω-3 polyunsaturated fatty acids include eicosapentaenoic acid (EPA), docosahexaenoic acid (DHA), eicosatetraenoic acid (ETA) and docosapentaenoic acid (DPA), most preferably at least EPA and/or DHA. EPA showed NFκB inhibition in the same in vitro model as used for the testing of oligosaccharides. The daily dosage of C20-C26 ω-3 polyunsaturated fatty acids can be between 200 mg-2000 g.

### EXAMPLE 1

Screening of oligosaccharides NFK-B inhibiting activity

| (Oligo)saccharide | NFκB inhibition |
|---|---|
| 1,1,1-kestopentaose | No |
| 1,1-kestotetraose | No |
| 1-kestose | No |
| Alginate | No |
| Amidated pectin | No |
| AOS from pectin | Yes |
| Apple pectin | Yes |
| Arabinogalactan | No |
| Arabinoxylan from rice bran | Yes |
| Digalacturonic acid | Yes |
| Fructose | No |
| Galactose | No |
| Galacturonic acid | No |
| Glucose | No |
| Iota-carrageenan II (irish moss) | No |
| Iota-carrageenan V (eucheuma spinosa) | No |
| Iota-carrageenan V (irish moss) | No |
| Maltodextrine | No |
| Palatinose | No |
| Polygalacturonic acid from citrus fruit | Yes |
| Polygalacturonic acid from oranges | Yes |
| Raffinose pentahydrate | No |
| Raftiline HP (long chain FOS) | No |
| Raftiline ST | No |
| Raftilose P95 (short chain FOS) | No |
| D-Ribose | Yes |
| β-D-glucan from barley | No |
| β-D-glucan from baker's yeast | Yes |
| Stachyose hydrate | No |
| Trigalacturonic acid | Yes |
| Vivinal-GOS | No |
| Λ-carrageenan (irish moss) | No |

### EXAMPLE 2

### Effect of Oligosaccharides in Combination with Cystein on NF-κB Inhibition

Methods: HEK293 cells were stably transfected with a NF-κB luciferase reporter gene construct. TNF-α was added to induce NF-κB activation. citrus pectin oligosaccharide (pAOS) was added 18 h prior to TNF-α addition (t=18 h). As a cystein source, NAC was added at the same time as TNF-α. At t=20.5 the production of luciferase was measured by chemiluminescence.

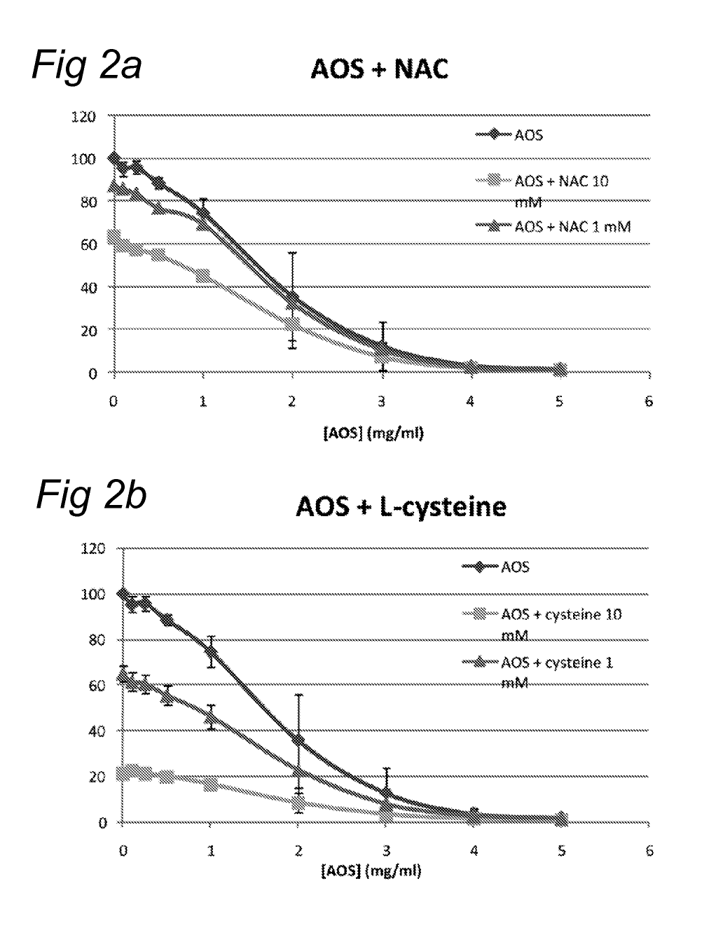

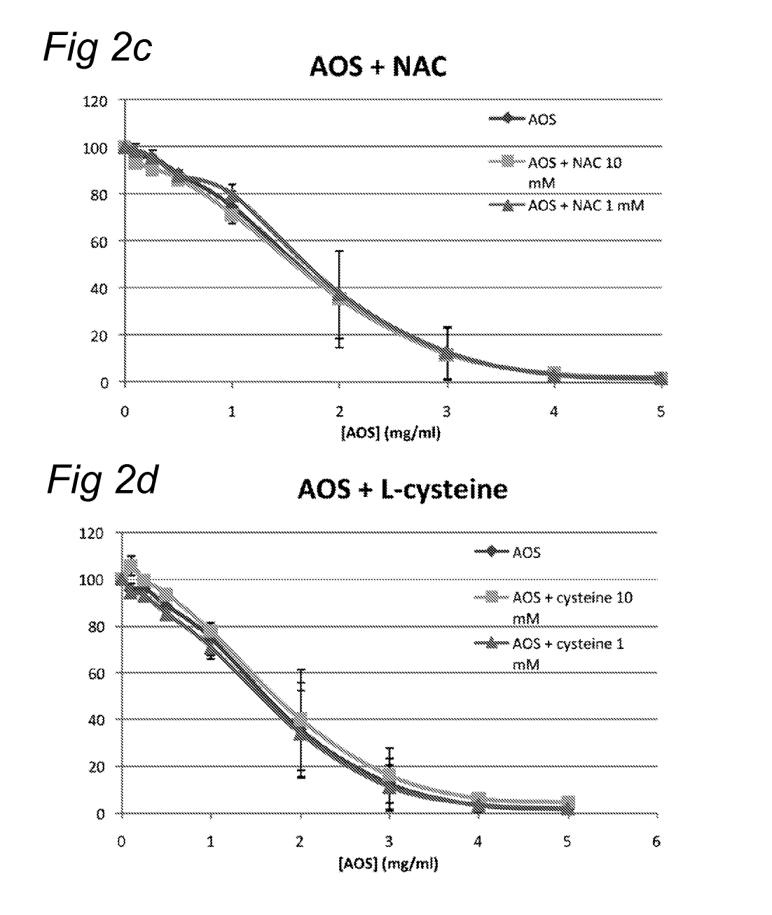

Results: Both pAOS and NAC inhibited TNF-α-mediated NF-κB activation dose dependently, although the timeframe of activity differed per component (FIGS. 2A and 2B). AOS-mediated NF-κB inhibition was optimal after an overnight pre-incubation period, while NAC-mediated inhibition was most effective after 2.5 hours. Upon correction for the contribution of NAC, the complete pAOS signal remained (FIGS. 2C and 2D). This finding suggests that the components do not interact with each other, but inhibit NF-kB activation in a different, additive way.

Conclusions: The addition of NAC to pAOS results in a strongly improved inhibition of NF-κB. The lack of competition between the two components, together with different incubation times required, suggest different working mechanisms for both components.

### EXAMPLE 3

### Effect of Oligosaccharides on HIV Production

Methods: HEK293 cells were calcium phosphate co-transfected with both a plasmid DNA to produce HIV-1 virus and a LAI construct containing either the CMV promoter or the phRL-TK promoter. HIV-1 virus concentrations were determined by measuring CA-p24 by an enzyme-linked immunosorbent assay. To correct inhibition of HIV-1 production for non specific inhibition of gene expression basal and TNF-alpha induced HIV-1 capside production was calculated relative to that of a co-transfected LAI construct, which was arbitrarily set at a value of 1. To control for NF-kappaB specificity of the inhibition, this co-transfected LAI construct contained either a promoter containing NF-kappaB binding sites (CMV promoter) or not (phRL-TK promoter). AOS was added immediately at transfection. Two days after transfection, the culture medium was removed and HIV viral production was measured by measuring CA-p24 by an enzyme-linked immunosorbent assay as described above. The cells were washed once with phosphate-buffered saline and lysed by the addition of 200 μl of reporter lysis buffer (Promega), and the sample was mixed for 45 min at room temperature. The lysate was collected in a tube, and the cell debris was removed by centrifugation for 15 min at 15,000 rpm in an Eppendorf centrifuge. The luciferase activity (in relative light units) was determined by a Berthold luminometer, model LB9501.

Results: All transiently co-transfected cells transfected with the complete HIV-1 genome combined with a control viral vector produced HIV-1 viral particles. This HIV-1 viral production was inhibited by AOS in all these cells. When the HIV-1 production was corrected for the activation of a promoter without NF-κB binding sites, however, production remained significant. Again, this production was inhibited by AOS. Moreover, production was stimulated by TNF-alpha. This TNF-alpha induced production was also inhibited by AOS. When the viral HIV production was corrected for a viral promoter containing NF-kappaB binding sites (CMV) (a positive control), hardly any activity remained.

HIV production [Ca-p24]/Renilla activity phRL-TK promoter

| | AOS concentration (mg/ml) | | | |
|---|---|---|---|---|
| | 0 | 1 | 2 | 3 |
| unstimulated | 2.4 ± 0.2 | 1.4 ± 0.1 | 0.7 ± 0.1 | 0.9 ± 0.1 |
| TNF stimulated | 8.0 ± 0.6 | 3.5 ± 0.5 | 1.4 ± 0.3 | 2.4 ± 0.2 |

HIV production when corrected for viral promoter activity of a viral promoter without NFkappaB binding sites.

HIV production [Ca-p24]/Renilla activity CMV

| | AOS concentration (mg/ml) | | | |
|---|---|---|---|---|
| | 0 | 1 | 2 | 3 |
| unstimulated | 3.0 ± 0.5 | 4.1 ± 0.8 | 4.8 ± 0.6 | 5.2 ± 0.5 |
| TNF stimulated | 3.7 ± 0.0 | 3.6 ± 0.0 | 3.4 ± 0.0 | 2.6 ± 0.0 |

HIV production when corrected for viral promoter activity of a viral promoter containing NFkappaB binding sites (CMV).

Conclusions: This study shows for the first time that specific dietary oligosaccharides (pAOS) are able to inhibit HIV-1 production in vitro. The HIV-1 production rate directly influences the HIV-1 replication rate. Moreover, the results from corrections with positive and negative controls (promoters containing NF-κB binding sites or not), demonstrate that it is a general mechanism that applies to other viruses with promoters containing NF-κB binding sites like CMV. Even more general, it is hypothesized that the components inhibit viruses that make use of NF-κB activation via different pathways for their replication.

### EXAMPLE 4

### Effect of Oligosaccharides in Combination with a Cystein Source on Glutathione

Methods: HEK293 cells were incubated with citrus pectin oligosaccharide (pAOS) for 18 h, then as a cystein source, NAC was added and incubated for 2.5 h. After the incubation period, the cells were centrifugated (2000 rpm for 5 min). Medium was discarded and taken up into ice-cold perchloric acid (0.4 M) and resuspended. Samples were then centrifuged for 10 min. at 13.000 rpm. Supernatant was collected and used for glutathione measurements. Total glutathione measurements were performed in 10 or 20 times diluted samples.

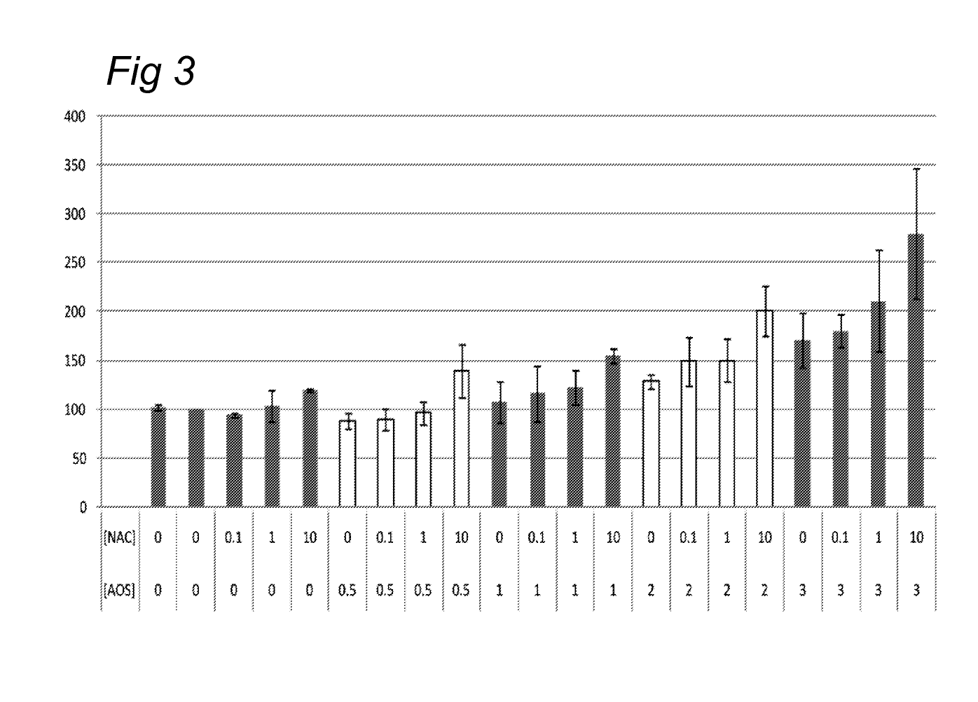

Results: Both pAOS and NAC inhibited TNF-α-mediated NF-κB activation dose dependently (FIG. 3), although the timeframe of activity differed per component. AOS-mediated NF-κB inhibition was optimal after an overnight pre-incubation period, while NAC-mediated inhibition was most effective after 2.5 hours. Upon correction for the contribution of NAC, the complete pAOS signal remained. This finding suggests that the components do not interact with each other, but inhibit NF-kB activation in a different, additive way.

Conclusions: The addition of NAC to pAOS resulted in a synergistic effect on glutathione. These results imply that disease states that show both an increase in oxidative stress and are related to infections with viruses that can be activated via NF-κB, might especially benefit from proposed combination. These disease are: COPD, IBD, Crohn, hepatitis and HIV (AIDS). E.g. Rhinovirus, the organism most often responsible for causing the common cold, is also the most common infectious cause of chronic obstructive pulmonary disease exacerbations. Coronavirus, influenza, respiratory syncytial virus, parainfluenza, adenovirus, and metapneumovirus are other important viral causes of chronic obstructive pulmonary disease exacerbations. Adenovirus increases acute exacerbations via NF-κB activated ICAM expression.

### EXAMPLE 5

### Effect of Oligosaccharides in Combination with L-Cysteine and EPA on NF-κB Inhibition

Methods: HEK293 cells were stably transfected with a NFκB luciferase reporter gene construct. One day after cell plating EPA (0, 25 μM) was added at t=0 h. pAOS (0, 1, 2 and 3 g/L) were added at t=96 h and L-cysteine (0, 1 mM) at t=120 h. NFκB activation was induced by 2.5 h incubation with TNF-α. Luciferase activity was subsequently assessed at t=122.5 h. As a control for cell viability cellular ATP levels were determined.

Results: The results are shown in the table below. Each component reduced NFκB activation in a dose dependent manner. When combined, EPA, L-cysteine and pAOS showed an additional inhibition of NFκB activation. There was no competition between the components added. Cellular ATP levels remained unchanged.

Conclusion: The combination of pAOS, L-cysteine and EPA showed an additive inhibition of NFκB activation in vitro. The lack of competition between the three components, together with different incubation times required, suggest different working mechanisms for all three components. pAOS alone inhibited HIV-1 production both in HEK293 cells and in SupT1 cells (see example 3). Since HIV-1 replication is mediated by NFκB, the present results on NFκB inhibition suggest that a combination of EPA, L-cysteine and pAOS can result in increased inhibitory effects on HIV-1 replication.

TABLE
Inhibition of NFκB activation

concentration AOS (mg/ml)

| | 0 | 1 | 2 | 3 |
|---|---|---|---|---|
| control | | | | |
| average | 100 | 72 | 40 | 24 |
| sd | 12 | 23 | 15 | 7 |
| 25 uM EPA | | | | |
| average | 113 | 62 | 29 | 8 |
| sd | 13 | 13 | 11 | 4 |
| 1 mM L-cysteine | | | | |
| average | 80 | 55 | 32 | 24 |
| sd | 14 | 12 | 8 | 6 |
| 25 uM EPA + L-cysteine | | | | |
| average | 83 | 45 * | 21 * | 8 |
| sd | 10 | 9 | 10 | 4 |

NFκB activation in HEK293-NFκB-Luc cells stimulated with TNFα was inhibited by pAOS. L-cysteine increased the inhibition of pAOS. EPA alone did not inhibit NFκB, but when combined with pAOS and L-cysteine it further enhanced the NFκB inhibition.

### EXAMPLE 6

### Effect of Oligosaccharides in Combination with a Cystein Source (L-Cysteine) and EPA on Glutathione

Methods: HEK293 cells were exposed to 25 uM EPA (t=0), one day after plating. HEK 293 cells were pre-exposed to 25 μM EPA for 96 hours. Then AOS (0, 1, 2, 3 mg/ml) was added and after 24 hours (t=120 hours), TNFα (50 ng/ml) and 1 mM L-cysteine was added. At t=122.5 h cells were resuspended in the media and glutathione assay was conducted. Total glutathione measurements were performed in 10 times diluted samples and levels were corrected for cell count.

Results: The results are shown in the table below. EPA (25 μM) alone did not affect GSH levels. When combined with AOS and L-cysteine, EPA increased GSH levels synergistically. By combining all three compounds, the increase of GSH levels was larger than the sum of the effects by the compounds alone.

Conclusions: The addition of EPA to L-cysteine and pAOS resulted in a synergistic effect on glutathione.

TABLE
Cellular glutathione levels.

| | concentration AOS (mg/ml) | | | |
|---|---|---|---|---|
| | 0 | 1 | 2 | 3 |
| control | | | | |
| average | 117 | 100 | 133 | 148 |
| sd | 21 | 18 | 40 | 58 |
| 25 uM EPA | | | | |
| average | 86 | 102 | 118 | 118 |
| sd | 27 | 20 | 38 | 57 |
| 1 mM L-cysteine | | | | |
| average | 104 | 116 | 194 | 229 |
| sd | 20 | 26 | 34 | 30 |
| 25 uM EPA + 1 mM L-cysteine | | | | |
| average | 117 | 142 | 234 | 329 * |
| sd | 66 | 82 | 63 | 56 |

Cellular glutathione levels in HEK293-NFkB-Luc cells were slightly elevated in presence of pAOS. EPA alone was not able to alter glutathione levels. L-cysteine combined with pAOS showed an additive increase in GSH levels. The combination of pAOS, L-cysteine and EPA was able to synergistically increase cellular glutathione levels.
* p < 0.05 compared to control.

1-17. (canceled)

18. A method for inhibiting viral replication in a mammal with a viral disease, comprising administering to the mammal a composition comprising polyuronic acid having an average degree of polymerization [DP] between 2 and 250 saccharide units, Arabinoxylan from rice bran, β-glucan from bakers yeast, D-Ribose, or mixtures thereof.

19. The method according to claim 18, wherein the polyuronic acid comprises pectin hydrolysates.

20. The method according to claim 19, wherein the pectin hydrolysates comprise digalacturonic acid, trigalacturonic acid and/or polygalacturonic acid.

21. The method according to claim 18, wherein the viral disease is selected from the group consisting of AIDS, HIV infection, cytomegalovirus infection, Adenovirus infection, Common cold (Rhino virus), Dengue fever, Entero virus infection, Hepatitis (A, B, C) infection, Herpes simplex infection, and Influenza (Flu) infection.

22. The method according to claim 21, wherein the viral disease is selected from the group consisting of AIDS, HIV infection, and cytomegalovirus infection.

23. The method according to claim 18, wherein the composition further comprises cystine, cysteine, cysteine salts, N-acetylcysteine or diacetylcysteine.

24. The method according to claim 18, wherein the composition further comprises ω-3 polyunsaturated fatty acids.

25. The method according to claim 24, wherein the comprising ω-3 polyunsaturated fatty acids comprise eicosapentaenoic acid (EPA).

26. The method according to claim 18, comprising co-administering an anti-viral medicament.

27. A nutritional composition comprising at least 20 en % protein, at least 5 en % fat, and at least 10 wt % based on dry weight of the composition, and at least one carbohydrate selected from the group consisting of polyuronic acid having an average degree of polymerization {DP] between 2 and 250 saccharide units, Arabinoxylan from rice bran, β-glucan from bakers yeast and D-Ribose.

28. The nutritional composition according to claim 27, further comprising at least one cysteine equivalent selected from the group consisting of cystine, cysteine, cysteine salts, N-acetylcysteine and diacetylcysteine.

29. The nutritional composition according to claim 27, wherein the polyuronic acid comprises pectin hydrolysates.

30. The nutritional composition according to claim 29, wherein the pectin hydrolysates comprise digalacturonic acid, trigalacturonic acid and/or polygalacturonic acid.

31. The nutritional composition according to claim 27, further comprising ω-3 polyunsaturated fatty acids.

32. The nutritional composition according to claim 31, wherein the ω-3 polyunsaturated fatty acids comprise at least eicosapentaenoic acid (EPA).

33. The nutritional composition according to claim 27, further comprising an anti-viral medicament.
